(19) United States
(12) Patent Application Publication
(10) Pub. No.: US 2022/0260595 A1
(43) Pub. Date: Aug. 18, 2022

(54) A BIOMARKER FOR ALZHEIMER'S DISEASE USING BLOOD SAMPLES FROM CLINICALLY DIAGNOSED ALZHEIMER'S DISEASE SUBJECTS

(71) Applicant: TODOS MEDICAL LTD., Rehovot (IL)

(72) Inventors: Elise BROWNELL, New York, NY (US); Paula TRZEPACZ, New York, NY (US); Gerald COMMISSIONG, New York, NY (US); Herman WEISS, Beit Shemesh (IL); Thomas ARENDT, Leipzig (DE)

(21) Appl. No.: 17/625,344

(22) PCT Filed: Jul. 10, 2020
(86) PCT No.: PCT/IB2020/056509

§ 371 Date: Jan. 7, 2022

Related U.S. Application Data
(60) Provisional application No. 62/872,567, filed on Jul. 10, 2019.

(51) Int. Cl. G01N 33/68

(52) U.S. Cl. CPC G01N 33/6896; G01N 2800/2821

(57) ABSTRACT

The current invention discloses a method for diagnosing Alzheimer disease by using biomarkers, and multivariate analysis which gives a more reliable, minimally- or non-invasive method of detection. The invention discloses simultaneous detection of CD69 protein in mitogenic lymphocytes, tau and phosphorylated tau proteins, and amyloid-β peptides in cerebrospinal fluid, which can replace or supplement conventional methods of detection of Alzheimer's disease such as cognitive testing and amyloid-positron emission tomography.

### FIELD OF INVENTION

The current invention relates to methods for diagnosing Alzheimer disease by using biomarkers, and by multivariate analysis which gives more reliable, non-invasive methods of detection. The invention discloses simultaneous detection of CD69 protein in mitogenic lymphocytes, tau and phosphorylated tau proteins, and amyloid-β peptides in cerebrospinal fluid, which can replace or supplement conventional methods of detection of Alzheimer's disease such as cognitive testing and amyloid-positron emission tomography.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND

Currently, an estimated 4.5 million Americans have Alzheimer's Disease (“AD”). The projected number of AD patients in US will be 11.3 million to 16 million by 2050. Moreover, neither Medicare nor most private health insurance covers the long-term care most patients need.

Alzheimer's Disease is a neurodegenerative disease of the central nervous system associated with progressive memory loss resulting in dementia. Two pathological characteristics are observed in AD patients at autopsy: extracellular plaques and intracellular tangles in the hippocampus, cerebral cortex, and other areas of the brain essential for cognitive function. Plaques are formed mostly from the deposition of amyloid beta (“Aβ”), a peptide derived from amyloid precursor protein (“APP”). Filamentous tangles are formed from paired helical filaments composed of neurofilament and hyperphosphorylated tau protein, a microtubule-associated protein. Late-onset/sporadic AD has virtually identical pathology to inherited early-onset/familial AD (FAD), thus suggesting common pathogenic pathways for both forms of AD. To date, genetic studies have identified three genes that cause autosomal dominant, early-onset AD, amyloid precursor protein (“APP”), presenilin 1 (“PS1”), and presenilin 2 (“PS2”). A fourth gene, apolipoprotein E (“ApoE”), is the strongest and most common genetic risk factor for AD, but does not necessarily cause it. All mutations associated with APP and PS proteins can lead to an increase in the production of Aβ peptides, specifically the more amyloidogenic form, Aβ₄₂. In addition to genetic influences on amyloid plaque and intracellular tangle formation, environmental factors (e.g., cytokines, neurotoxins, etc.) may also play important roles in the development and progression of AD.

The main clinical feature of AD is a progressive cognitive decline leading to memory loss. The memory dysfunction involves impairment of learning new information which is often characterized as short-term memory loss. In the early (mild) and moderate stages of the illness, recall of remote well-learned material may appear to be preserved, but new information cannot be adequately incorporated into memory. Disorientation with respect to time is closely related to memory disturbance.

Language impairments are also a prominent part of AD. These are often manifest first as word finding difficulty in spontaneous speech. Complex deficits in visual function are present in many AD patients, as are other focal cognitive deficits such as apraxia, acalculia and left-right disorientation. Impairments of judgment and problems solving are frequently seen.

Non-cognitive or behavioural symptoms are also common in AD and may account for an event larger proportion of caregiver burden or stress than the cognitive dysfunction. Currently, the primary method of diagnosing AD in living patients involves taking detailed patient histories, administering memory and psychological tests, and ruling out other explanations for memory loss, including temporary (e.g., depression or vitamin B₁₂ deficiency) or permanent (e.g., stroke) conditions. These clinical diagnostic methods, however, are not fool proof.

However, because AD is only one of seventy conditions that produce dementia, AD cannot be diagnosed with complete accuracy until after death, when autopsy reveals the disease's characteristic amyloid plaques and neurofibrillary tangles in a patient's brain. In addition, clinical diagnostic procedures are only helpful after patients have begun displaying significant, abnormal memory loss or personality changes. By then, a patient has likely had AD for years.

Positron emission tomography (PET) associated with various molecular imaging agents reveals numerous aspects of dementia pathophysiology, such as brain amyloidosis, tau accumulation, neuroreceptor changes, metabolism abnormalities and neuroinflammation in dementia patients. In the context of a growing shift toward presymptomatic early diagnosis and disease-modifying interventions, PET molecular imaging agents provides an unprecedented means of quantifying the AD pathophysiological process, monitoring disease progression, ascertaining whether therapies engage their respective brain molecular targets, as well as quantifying pharmacological responses. Yet, PET methods are expensive and stressful for the patients. Due to radioactivity safety concerns it is generally recommended to wait about 12 months before reimaging a patient with amyloid PET using approved radiotracers. PET scanning requires a nuclear medicine center capable of producing and/or handling the radionuclide tracer and, of course, requires non-ubiquitous PET scan equipment, radioactivity-protected suite and staffing. Amyloid PET scans are available in mostly only in large metropolitan centers and are expensive.

Given the magnitude of the public health problem posed by AD, considerable research efforts have been undertaken to elucidate the etiology of AD as well as to identify biomarkers (secreted proteins or metabolites) that can be used to diagnose and/or predict whether a person is likely to develop AD. The proteins amyloid beta and tau are probably the most well characterized. Cerebrospinal fluid (“CSF”) samples from AD patients contain higher than normal amounts of tau, which is released as neurons degenerate, and lower than normal amounts of beta amyloid, presumably because it is trapped in the brain in the form of amyloid plaques. Because these biomarkers are released into CSF, a lumbar puncture (or “spinal tap”) is required to obtain a sample for testing. Thus it is highly desirable to have one biomarker, or a group of biomarkers which have strong correlation to AD, and can be early and minimally- or non-invasive methods of diagnosis and treatment efficacy.

### SUMMARY

The current invention relates to methods for diagnosing Alzheimer disease by using biomarkers, and by multivariate analysis which gives more reliable, non-invasive method of detection. The invention discloses simultaneous detection of CD69 protein in mitogenically stimulated lymphocytes, tau and phosphorylated tau proteins, and amyloid-β peptides in cerebrospinal fluid, which can replace or supplement conventional methods of detection of Alzheimer's disease such as cognitive testing and amyloid-positron emission tomography.

One embodiment of the invention is a method of detecting Alzheimer's disease (AD) in a human subject without amyloid Positron Emission Tomography (PET) imaging, the method comprising the steps of: (a) obtaining a sample from the subject; (b) comparing normalized measured level of at least four biomarkers from the subject's sample to a reference level of each AD diagnosis biomarker, wherein the four biomarkers are CD69, tau, phosphorylated-tau proteins and amyloid-β peptide; and wherein the reference level of each AD diagnosis biomarker comprises a normalized measured level of the AD diagnosis biomarker from one or more samples of patients with confirmed AD and wherein levels of tau proteins are less in the subject's sample than the reference level , and the levels of p-tau and amyloid-β peptide are greater in the subject's sample than in the reference level.

In one embodiment, the diagnosis of AD in the subject is confirmed by amyloid PET imaging.

In one embodiment, the level of CD69 protein is measured in mitogenic lymphocytes from peripheral blood samples.

In one embodiment, the tau, p-tau protein and amyloid-β peptide levels are measured in cerebrospinal fluid (CSF) samples.

In one embodiment, there is a high inverse correlation between the positive PET imaging result of AD and expression of CD69 protein in mitogenic lymphocytes.

In one embodiment, there is high inverse correlation between positive PET imaging result of AD result and expression of tau protein.

In one embodiment, there is high positive correlation between the positive PET imaging result of AD result and expression of p-tau protein.

In one embodiment, there is high positive correlation between the positive PET imaging result of AD result and expression of amyloid-β peptide.

In one embodiment, the method disclosed herein is used for selecting subjects for AD treatment.

In one embodiment, the method is used for determining treatment efficacy in subjects undergoing treatment for AD.

In one embodiment, it is used for severity assessment of AD in a subject by determining correlation between PET imaging results and expression of CD69 protein in mitogenic lymphocytes from a subject.

In one embodiment, the method is used for determining AD progression in a human subject.

In one embodiment, the CD69 levels in mitogenic lymphocytes are assessed by applying the lymphocyte activation score in LymPro assay.

In one embodiment, the method comprises multivariate analysis of five variables including the LymPro activation score, expression of tau protein in CSF and PET imaging, expression of phosphorylated-tau protein in CSF and PET imaging, expression of amyloid-β peptide in CSF, and amyloid-PET imaging.

In one embodiment, the method is used to determine the risk of developing AD.

In one embodiment, the method is used to distinguish AD-associated dementia from other forms of dementia.

In one embodiment, the method can be used to discover new potential therapeutic agents that can normalize or reverse cell cycle dysfunction as measured by CD69 expression.

### BRIEF DESCRIPTION OF THE DRAWINGS

Novel features of the present invention are set forth below. A better understanding of the features and advantages of the invention will be obtained by reference to the following detailed description that sets forth illustrative embodiments, in which the principles of the invention are utilized, and the accompanying drawings of which:

FIG. 1 shows a review of the cell cycle. G1 gap phase 1; S—synthesis phase; G2 gap phase 2; M—mitotic phase; G₀—quiescent state.

FIG. 2 illustrates white blood cell (WBC) subtypes and also illustrates the five WBC subtypes (enclosed by hatched lines) that were studied and analyzed by flow cytometry.

FIGS. 3A and 3B shows Receiver Operating Characteristic (ROC) curves for the Alzheimer's disease (AD) and healthy control (HC) groups for results of the LymPro assay (n=125). FIG. 3A shows the ROC curves for the training set (n=81; AD=38, HC=43); FIG. 3B shows the ROC curves for the test set (n=44; AD=21, HC=23), all from our completed multicenter proof of concept study. Area under the curve (AUC) values are also shown in the tables.

FIG. 4 shows interim analysis results of an ongoing study for one of the lymphocyte stimulation paradigms. The table presents these interim analysis results for 20 patients with Mild Cognitive Impairment (MCI) or dementia due to AD who had a positive amyloid PET scan (clinically read) (anonymized, as captioned, in the left-most table column; gender indicated), for test values of parameters indicated in column-headers of:
All subjects (ID designation and gender);
LymPro B-cell CD69 activation results (test date indicated);
Amyloid PET scan quantitative measurements expressed as the composite standardized uptake value ratio (cSUVR) of the radiotracer used (either ¹⁸F-Florbetaben (FBB) or ¹¹C-Pittsburgh compound B (PiB), as indicated) to image the density of brain β-amyloid plaque (raw data, unadjusted for radionuclide species used);
the cSUVR values for subjects receiving PiB, adjusted to harmonize results across the different radionuclides (for ¹⁸F, values are for the raw data); and
MiniMental State Exam (MMSE) cognitive function scores.
   For several subsets of the 20 patients, values are also shown for relationships between the LymPro assay and cerebrospinal fluid (CSF) concentrations of tau, phosphorylated tau (P-tau) and/or amyloid-β proteins; and/or apolipoprotein E genotype.

FIG. 5 shows results of the interim analysis of the ongoing amyloid PET study for which datasets of FIG. 4 are plotted against each other, the graph showing correlation between LymPro B-lymphocyte CD69 activation scores against amyloid PET adjusted cSUVR values for all 20 patients.

FIG. 6 shows results of the interim analysis of the ongoing study in which datasets of FIG. 4 are plotted against each other, the graph showing correlation between LymPro CD69 activation scores (for B-lymphocyte) against amyloid PET scan raw cSUVR values for the subset of patients receiving ¹¹C-PiB.

FIG. 7 shows results of the interim analysis of the ongoing amyloid PET study for which specific indicated datasets of FIG. 4 are plotted against the LymPro CD69 activation score data (LymPro scores adjusted to harmonize results across the different radionuclides), with a summary table of results.

### DETAILED DESCRIPTION OF THE INVENTION

### INTRODUCTION

The Lymphocyte Proliferation Test (LymPro®) is a blood assay that has reported differential mitogenic activation in peripheral lymphocytes drawn from the blood of clinically-diagnosed Alzheimer's disease (AD) subjects as compared to healthy controls (HC).

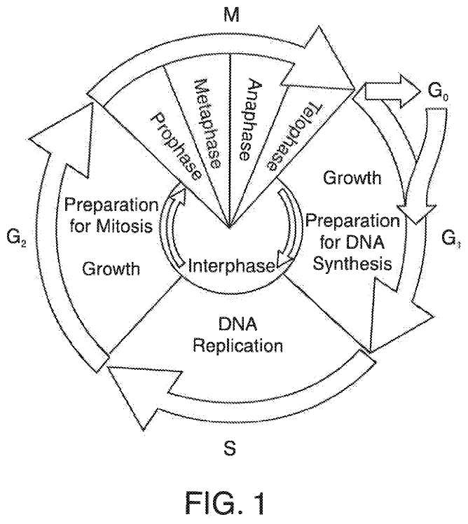

The assay is based on the cell cycle reentry hypothesis for AD, which states that post-mitotic central nervous system (CNS) neurons in AD have inappropriately reentered the cell cycle, as evidenced by histology revealing downstream overexpression of cytokines, cell cycle related proteins, DNA polyploidy, and increased neuronal cell death through apoptosis (Yang et al 2003; Herrup 2012). (See FIG. 1.) Mature neurons resting in the G₀ state should not re-enter the cell cycle (e.g., go past the G1/S checkpoint) but sometimes they do, which is evidence of cell cycle dysregulation (CCD). This CCD is likely one of the earliest key neuropathologies in AD and also appears to be linked to tau hyperphosphorylation and amyloid precursor protein (APP) metabolism (Seward et al 2013).

Brain CCD appears to be reflected by systemic manifestations, reported as CCD measured in stimulated white blood cells by several research groups. Dr. Thomas Arendt et al. at Leipzig University developed the specific technique for measuring white blood cells' expression of CD69 as an indicator of Alzheimer's disease. CD69 is a cell surface receptor that serves as a marker of activation of lymphocytes' entry into the cell cycle for proliferation. When stimulated by an antigen or a nonspecific mitogen, lymphocytes normally enter the cell cycle and a marker of their passing the G1/S checkpoint is an increased expression of CD69, which can be measured by flow cytometry, whereas in AD patients' CD69 does not increase indicating some derangement of the regulation of the normal cell cycle (see FIG. 1).

We used this in vitro assay in lymphocytes and monocytes obtained from AD and HC subjects to further develop an adjunctive diagnostic test useful as a blood biomarker to increase the accuracy of clinical diagnosis of cognitive impairment attributable to AD. Having a highly accurate peripheral blood-based biomarker of AD would be very desirable for physicians, patients and their families who would like to know the underlying etiology for cognitive impairment.

### Embodiments

One embodiment of the invention is a method of detecting Alzheimer's disease (AD) in a human subject without amyloid Positron Emission Tomography (PET) imaging, the method comprising the steps of: (a) obtaining a sample from the subject; (b) comparing normalized measured level of at least four biomarkers from the subject's sample to a reference level of each AD diagnosis biomarker, wherein the four biomarkers are CD69, tau, phosphorylated-tau proteins and amyloid-β peptide; and wherein the reference level of each AD diagnosis biomarker comprises a normalized measured level of the AD diagnosis biomarker from one or more samples of patients with confirmed AD and wherein levels of tau proteins is less in the subject's sample than the reference level , and the level of p-tau and amyloid-β peptide is greater in the subject's sample than in the reference level.

In one embodiment, the diagnosis of AD in the subject is confirmed by amyloid PET imaging.

In one embodiment, the level of CD69 protein is measured in mitogenic lymphocytes from peripheral blood samples.

In one embodiment, the tau, p-tau protein and amyloid-β peptide levels are measured in cerebrospinal fluid (CSF) sample.

In one embodiment, there is high inverse correlation between the positive PET imaging result of AD and expression of CD69 protein in mitogenic lymphocytes.

In one embodiment, there is high inverse correlation between positive PET imaging result of AD result and expression of tau protein.

In one embodiment, there is high positive correlation between the positive PET imaging result of AD result and expression of p-tau protein.

In one embodiment, there is high positive correlation between the positive PET imaging result of AD result and expression of amyloid-β peptide.

In one embodiment, the method disclosed herein is used for selecting subjects for AD treatment.

In one embodiment, the method is used for determining treatment efficacy in subjects undergoing treatment for AD.

In one embodiment, it is used for severity assessment of AD in a subject by determining correlation between PET imaging results and expression of CD69 protein in mitogenic lymphocytes from a subject.

In one embodiment, the method is used for determining AD progression in a human subject.

In one embodiment, the CD69 levels in mitogenic lymphocytes are assessed by the lymphocyte activation score in the LymPro assay.

In one embodiment, the method comprises multivariate analysis of five variables including LymPro activation score, expression of tau protein in CSF, expression of phosphorylated-tau protein in CSF, expression of amyloid-β peptide in CSF, and amyloid-PET imaging.

In one embodiment, the method is used to determine the risk of developing AD.

In one embodiment, the method is used to distinguish AD-associated dementia from other forms of dementia.

In one embodiment, the method can be used to discover new potential therapeutic agents that can normalize or reverse cell cycle dysfunction as measured by CD69 expression.

In one embodiment, the method is used to enrich for rare mitogenic lymphocyte subsets to assess the level of cell cycle dysfunction as measured by CD69 expression (e.g., group 2 innate lymphocytes).

In one embodiment, the method is used to discover new agents that can improve or normalize cell cycle dysfunction as assessed by CD69 expression.

### EXAMPLES

### METHODS OF COMPLETED MULTISITE PROOF OF CONCEPT LP202 STUDY

Subjects were diagnosed as having AD clinically by dementia experts using NIA/AA (2011) clinical criteria for the determination of probable Alzheimer's dementia. Table 1 details the demographics of the 125 subjects. Table 2 shows the MMSE scores for subjects in the AD group.

TABLE 1
Demographics

| Variable | All | AD | HC | p* |
|---|---|---|---|---|
| N | 125 | 59 | 66 | |
| Mean Age | 73.1 ± 9.6 | 77.2 ± 9.0 | 69.6 ± 7.8 | <0.0001 |
| Gender (M/F) | 50/75 | 26/33 | 24/42 | 0.02 |
| APOE-4 status (+/−) | 44/81 | 28/31 | 16/50 | 0.009 |

*p values between AD & HC groups

TABLE 2
MMSE in the AD Cohort (HC was ≥28)

| | |
|---|---|
| N | 59 |
| Mean MMSE ± SD | 16.1 ± 5.5 |
| Range | 0-26 |

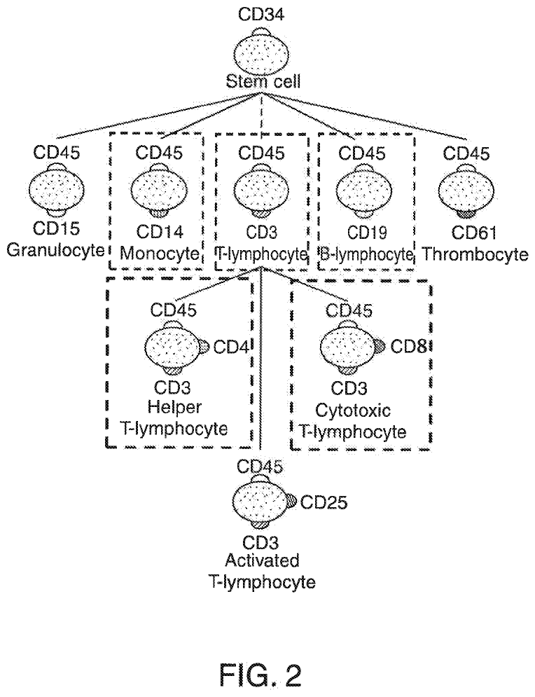

Lymphocyte Proliferation Assay Procedure: Whole blood samples were drawn from 141 enrolled study participants in vacutainer tubes designed for lymphocyte culture. Samples were shipped over night, cultured with or without mitogen (PHA or PWM) in separate culture tubes, and then stained with an antibody cocktail to reveal subpopulations of lymphocytes (T, B, and monocytes) as well as expression levels of CD69 (a surface marker of cell cycle activity) cell stimulation expression. Lymphocyte subpopulation specific biomarkers were measured on an 8-color flow cytometer at a contract lab (Becton Dickinson). After analytical review of the quality of samples for flow cytometry, n=125 of the samples passed blinded quality control (59 AD and 66 controls) and were used for the study analyses. Each subject's WBCs were characterized by measuring 14 biomarker identification features (see FIG. 2) in various permutations for statistical analyses, as well as two stimulation indices that were calculated, to produce an additional 8 biomarker variables. Thus, results of 22 variables were analyzed statistically. These 22 were measured for each of three stimulation conditions using mitogens.

Statistical Analysis: Public domain feature selection algorithms or stepwise methods were used to identify optimal feature sets to maximize diagnostic prediction performance. These included logistic regression, discriminant analysis (linear and quadratic), and decision and random forest methods. Prediction performance was initially assessed with a 65% training set (n=81) and applied to a 35% test set (n=44). All analysis was done in JMP Pro v11.2.1 (SAS, Cary, NC).

### MULTIVARIATE RESULTS OF LP202 STUDY

Of the 66 variables, 5 were selected in multivariate analysis as together providing the best differentiation between groups.

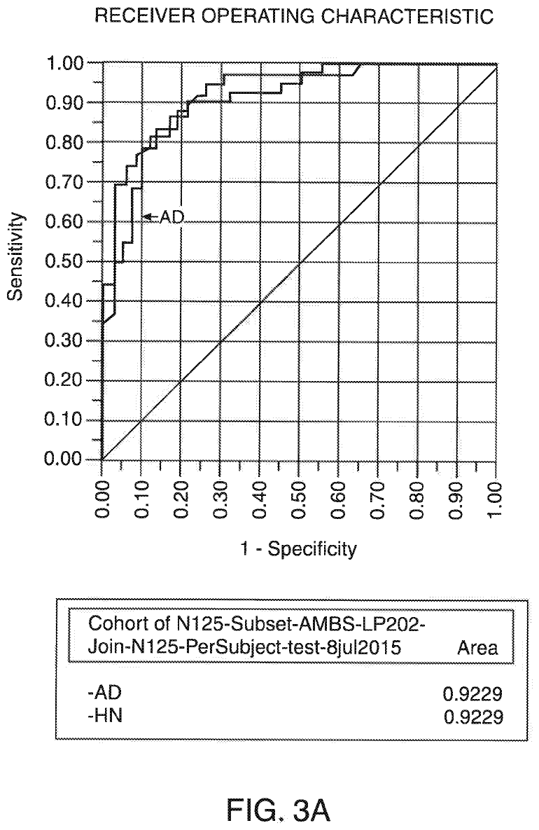

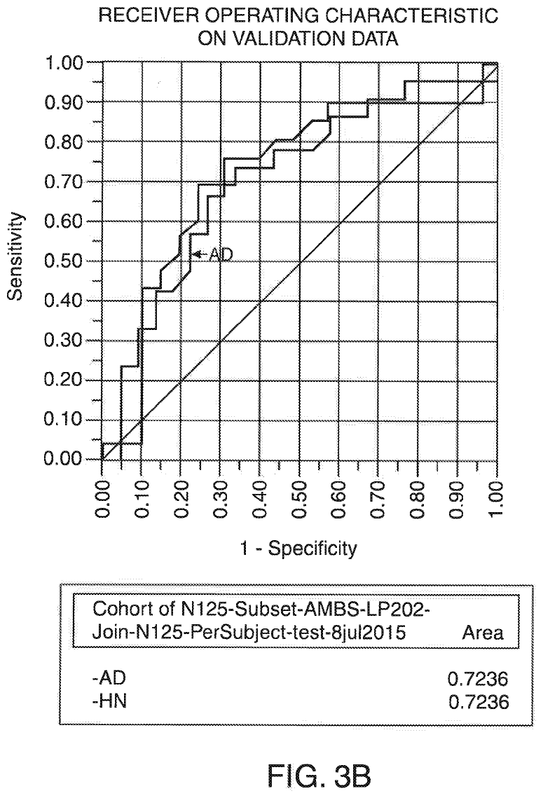

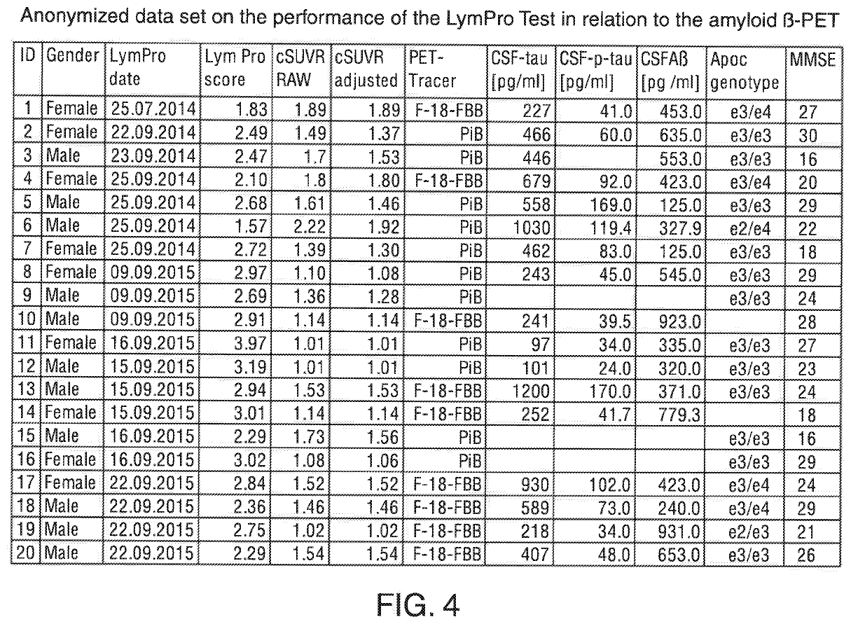

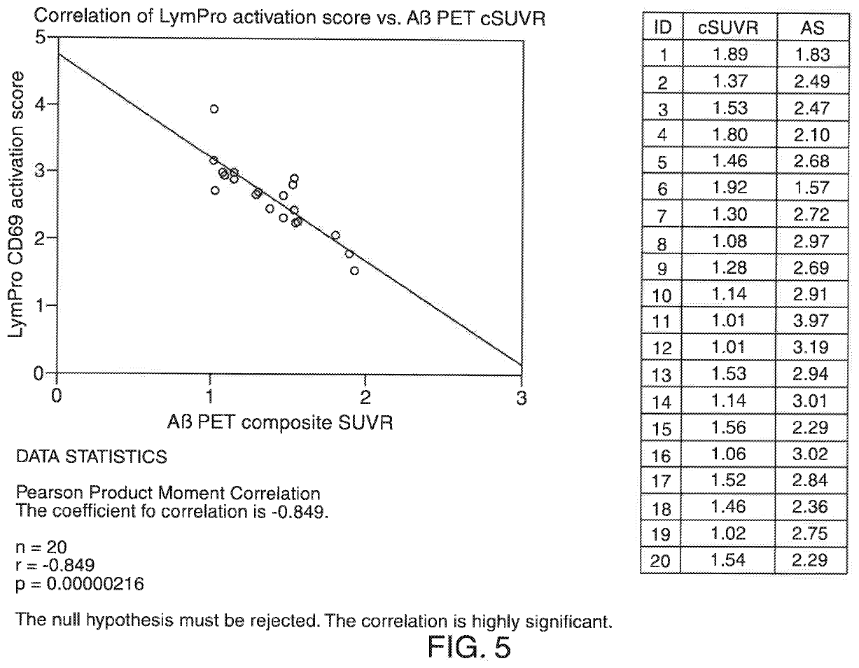

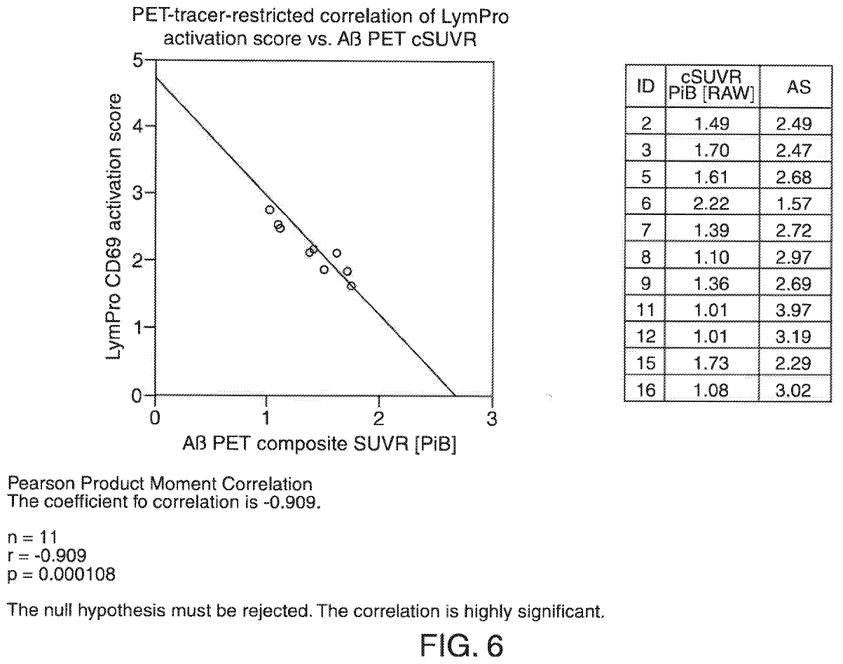

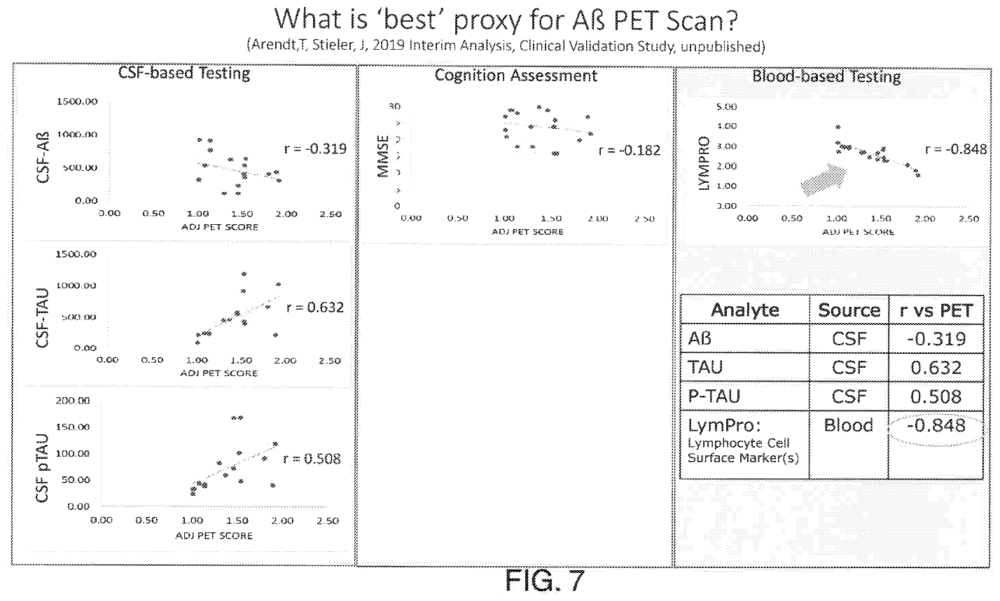

ROC graphs were produced using these 5 candidate features for training and test data sets (see FIG. 3) where the AUCs for AD and HC groups were good to excellent.

It is notable that all five candidate features were obtained from the same mitogen stimulation condition.

### DISCUSSION OF PREVIOUS STUDY

Findings from this expanded analysis of the Lymphocyte Proliferation test using multivariate analysis are consistent with the two prior published reports using univariate approaches. This lent further support to the proposal that the Lymphocyte Proliferation test may have utility as a blood biomarker reflective of a key AD neuropathology, cell cycle dysfunction. These findings in conjunction with subsequent in-depth reanalyses of the previously published study cohorts are encouraging and warranted continued research to further define the test validation and thereby demonstrate the utility of this blood biomarker assay in the differential diagnosis of patients with cognitive impairment.

### LIMITATIONS OF PREVIOUS STUDIES

The study designs were predicated upon cohort characterization and categorization (AD or HC) on clinical grounds only and there were no AD-specific adjunctive biomarker tests employed for confirmation of clinical diagnosis.

Nonetheless, the completed LP202 proof of concept test sample performed similarly to Beach et al's (2012) conclusion about clinical diagnosis of AD that “... when optimizing for sensitivity and specificity, the best [clinical] result was 70.9% sensitivity and 70.8% specificity.”

### OVERALL CONCLUSIONS OF PREVIOUS STUDIES USING ONLY CLINICAL DIAGNOSIS AS THE GOLD STANDARD

1: Multivariate analysis using random forest found 5 candidate lymphocyte variables that together generated the best performance results in ROC analysis.

2: This preliminary algorithm held promise as a step in the development of a bioassay algorithm that can yield both strong sensitivity and specificity.

3: Though the Lymphocyte Proliferation test as used in previous studies showed promise for use in evaluating patients, further clinical validation was needed including in more diverse clinical samples and in conjunction with clinical diagnosis aided by an autopsy surrogate biomarker, amyloid PET scans, as carried out in currently ongoing studies presented below. As will be shown, the results of the current studies exceed test performance expectations of prior studies.

### INTRODUCTION TO CURRENT PET STUDY

Amyloid PET uses one of several available radiotracers specific to radioactively labelling β-amyloid plaque in the brain to measure its density and location. It is a valid surrogate marker of AD as diagnosed by autopsy, with a very high accuracy. It allows antemortem biological support for a clinical diagnosis. A recently published report from the IDEAS naturalistic study (Rabinovici et al 2019) found greatly increased diagnostic accuracy using amyloid PET with a reduced misdiagnosis rate of patients. By studying the Lymphocyte Proliferation (“LymPro”) test in MCI and dementia patients whose diagnosis of AD was made in conjunction with amyloid PET brings high confidence as compared to making a clinical diagnosis without a specific AD biomarker.

### DESIGN AND METHODS OF CURRENT STUDIES

LymPro® Assay Procedure: The LymPro® Assay Procedure was carried out as described for the proof of concept study.

Amyloid PET scans were performed using either ¹⁸F-FBB or ¹¹C-PiB radiotracers. Scans of decay-corrected brain radioactivity concentration were obtained of each subject's frontal, parietal, lateral temporal, anterior and posterior cingulate, and occipital cortices, yielding a composite scan also normalized for injected dose and body-mass. From this, the subject's raw composite standardized uptake value ratio (cSUVR) was calculated. An adjusted cSUVR normalizing raw ¹¹C-PiB-results to raw ¹⁸F-FBB results was also calculated for subjects scanned using ¹¹C-PiB.

For a subset (16/20) of the cohort, cerebrospinal fluid (CSF) was obtained by lumbar puncture, and the samples were assayed for concentration of tau-protein (CSF-tau), hyperphosphorylated tau-protein (CSF-p-tau) and amyloid-β peptides (CSF Aβ). For another large subset (18/20), allelic genotype was determined for apolipoprotein E (ApoE genotype), implicated in amyloid precursor protein metabolism, with each of the two alleles being one of normal “neutral” type ε3 or variant ε2 (possibly Parkinson's-implicated) or variant ε4 (implicated in AD, other cognitive impairment, as well as in other disorders such as multiple sclerosis and atherosclerosis).

In this ongoing LymPro® and amyloid PET study, 20 subjects were recruited who had been evaluated at the University of Leipzig Memory Disorders Clinic (in Germany) and diagnosed as having MCI or dementia due to AD by experts using clinical criteria for the determination of probable AD as well as having a positive amyloid PET brain scan for a more definitive diagnosis as being positive for AD. Eleven were male and 9 female, whose mean age was 70.4 years (±8.9). Their MMSE scores ranged from 16-30.

Data/statistics processing Correlation graphing and statistics were performed by standard public domain mathematics software, such as SigmaPlot.

### DISCUSSION OF ONGOING STUDY INTERIM ANALYSIS

The correlations displayed in the plots in FIGS. 5 and 6 of FIG. 4 data show very strong inverse correlations between LymPro® results with the PET cSUVR values in the interim analysis of 20 patients with positive PET scans and AD. Amyloid PET neuroimaging has become accepted as providing highly accurate adjunctive diagnostic information during the differential diagnosis of AD, however it requires production of the radiotracer in a nearby cyclotron and infusion of radiotracer prior to being imaged by a PET scan machine.

Correlation between LymPro® scores and MMSE scores (not shown) was very low, as was the correlation between MMSE and amyloid PET cSUVR. Cognitive assessments do not reveal etiology of impairments and are not biomarkers. Therefore, the MMSE should not be expected to correlate strongly with amyloid PET results. Degree of cognitive impairment can inform staging of disease which may or may not be associated with more specific biomarker values. Even an objective biological criterion such as apolipoprotein E genotype is only weakly predictive and of limited diagnostic utility. Thus, while results of the previous clinical diagnosis-based studies comparing LymPro® scores with outcomes of cognitive function testing were suggestive of the utility of LymPro® as an AD diagnostic tool, the correlations shown in the FIGS. 5 and 6 plots of −0.849 (p=0.00000216) and −0.909 (p=0.000108), respectively, strongly recommend LymPro® as a true adjunctive AD diagnostic tool. It would be expected to have an inverse correlation with cSUVR because more cell cycle dysfunction would be more strongly related to greater β-amyloid plaque burden.

Comparison of the correlation coefficients of the amyloid PET cSUVR study data with the CSF biomarkers study data underscore the utility of LymPro® as a true adjunctive AD diagnostic tool. The FIG. 5 correlation coefficient with raw PET of LymPro® is −0.849. As shown in FIG. 7, correlation analysis of the non-PET data against the adjusted PET data shows correlation coefficients with PET of the CSF biomarkers tau, phosphorylated tau and amyloid-β of −0.319, 0.632 and 0.508, respectively; of the MMSE study data, a correlation coefficient of −0.182; and with the LymPro® data, a correlation coefficient of −0.848.)

### SUMMARY CONCLUSIONS OF CURRENT PET STUDY

Given the strong correlations of LymPro® with PET results, other advantages of LymPro may make it a highly useful adjunctive test for AD diagnosis. Due to radioactivity safety concerns it is generally recommended to wait about 12 months before reimaging a patient with amyloid PET using approved radiotracers. From the patient's perspective LymPro® involves a simple drawing of a venous blood sample which is widely available. PET scanning requires a nuclear medicine center capable of producing and/or handling the radionuclide tracer and, of course, requires non-ubiquitous PET scan equipment, radioactivity-protected suite and staffing. Amyloid PET scans are available in large metropolitan centers and are expensive. Once again, LymPro® offers itself as a convenient sampling protocol from a doctor's office or a phlebotomist-staffed clinical sample-collecting service.

LymPro® as an adjunctive AD diagnostic tool may, therefore, be of utility not only in diagnosing but also potentially monitoring the onset and progression of AD. With the rate over time of disease progression potentially readily, sensitively and economically monitored by using the LymPro® test, changes in that rate might be used to closely follow disease response to specific therapies and relative to patients' other health issues.

The current studies point to the utility and sensitivity of readily harvested immune cells in mirroring CNS anatomical/physiological realia. Work on other immune cell-based tests for AD diagnosis and a variety of other neurodegenerative diseases, such as Parkinson's, traumatic brain injury, chronic traumatic encephalopathy (CTE), frontotemporal dementia, multiple sclerosis, and so on are also being pursued. Preliminary work has revealed LymPro distinguishes patients with AD dementia from those with Parkinson's dementia, whose results were comparable to that of healthy controls (Stieler et al 2012).

Some of these other tests include using immune cell types besides CD69 as markers of neurodegenerative disease, with particular evaluation of the utility of cells expressing CD4 and CD19. Additionally, as indicated by the collection of data of CSF protein concentrations of tau, hyperphosphorylated tau and amyloid-β proteins shown in FIG. 4, work is underway on evaluating their correlation to PET scans and to LymPro®. Also, being explored is the use of Fourier Transform IR (FTIR) micro spectroscopy as a modality to measure cell cycle cell cycle dysregulation.

### ASSAY VALIDATION STUDY

We have performed an analytical validation at ICON to assess optimal antibody concentrations, inter- and intra-assay precision, and analyst-analyst and instrument-instrument reproducibility.

https://amarantusbioscienceholdings.box.com/s/fmn51vnih4q0z8an87fmheavyxbmxbbrg

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

We claim:

1. A method of detecting Alzheimer's disease (AD) in a human subject without amyloid Positron Emission Tomography (PET) imaging, the method comprising the steps of:
a. obtaining a sample from the subject;
b. comparing normalized measured level of at least four biomarkers from the subject's sample to a reference level of each AD diagnosis biomarker, wherein the four biomarkers are CD69, tau, phosphorylated-tau proteins and amyloid-β peptide; and wherein the reference level of each AD diagnosis biomarker comprises a normalized measured level of the AD diagnosis biomarker from one or more samples of patients with confirmed AD and wherein levels of tau proteins is lesser in the subject's sample than the reference level, and the level of p-tau and amyloid-β peptide is greater in the subject's sample than in the reference level.

2. The method of claim 1, wherein the diagnosis of AD in the subject is confirmed by amyloid PET imaging.

3. The method of claim 1, wherein CD69 protein is measured in mitogenic lymphocytes from peripheral blood samples.

4. The method of claim 1, wherein tau, p-tau protein and amyloid-β peptide levels are measured in cerebrospinal fluid (CSF) sample.

5. The method of claim 3, wherein there is high inverse correlation between the positive PET imaging result of AD and expression of CD69 protein in mitogenic lymphocytes.

6. The method of claim 4, wherein there is high inverse correlation between positive PET imaging result of AD result and expression of tau protein.

7. The method of claim 2, wherein there is high positive correlation between the positive PET imaging result of AD result and expression of p-tau protein.

8. The method of claim 2, wherein there is high positive correlation between the positive PET imaging result of AD result and expression of amyloid-β peptide.

9. The method of claim 1, wherein is used to determine the risk of developing AD.

10. The method of claim 1, wherein it is used for selecting subjects for AD treatment.

11. The method of claim 1, wherein the method is used for determining treatment efficacy in subjects undergoing treatment for AD.

12. The method of claim 1, wherein it is used for severity assessment of AD in a subject by determining correlation between PET imaging results and expression of CD69 protein in mitogenic lymphocytes from a subject.

13. The method of claim 1, wherein it is used to distinguish AD-associated dementia from other forms of dementia.

14. The method of claim 1, wherein it is used to discover novel therapeutic agents to reverse cell cycle dysfunction as measured by CD69 expression.

15. The method of claim 1, wherein the method is used for determining AD progression in a human subject.

16. The method of claim 1, wherein the CD69 levels in mitogenic lymphocytes are assessed by the lymphocyte activation score in the LymPro® assay.

17. The method of claim 13, wherein the method comprises multivariate analysis of five variables including the LymPro® activation score, expression of tau protein in CSF, expression of phosphorylated-tau protein in CSF, expression of amyloid-β peptide in CSF, and amyloid-PET imaging.

18. The method of claim 1, wherein it is used to enrich for rare mitogenic lymphocyte subsets to assess the level of cell cycle dysfunction as measured by CD69 expression.
